(19) United States
(12) Patent Application Publication
(10) Pub. No.: US 2026/0215826 A1
(43) Pub. Date: Jul. 30, 2026

(54) DEVICE FOR OSTEOSYNTHESIS

(71) Applicant: TSQ GLOBAL S.R.L., Cantarana (AT) (IT)

(72) Inventor: Gianluca TORDI, Derendingen (CH)

(21) Appl. No.: 19/147,565

(22) PCT Filed: Jan. 19, 2023
(86) PCT No.: PCT/IB2023/050465

§ 371 Date: Jul. 11, 2025

(51) Int. Cl. A61B 17/80; A61B 5/00; A61B 17/00

(52) U.S. Cl. CPC A61B 17/80; A61B 5/686; A61B 2017/0011; A61B 2017/00221; A61B 2017/00526

(57) ABSTRACT

It is provided a device (1) for osteosynthesis configured to support the healing of an osseous fracture comprising a main structure (2) defining a expansion surface (2a), a plate (20) extending predominantly along the expansion surface (2a), fastening elements (3) suitable for constraining integrally the plate (20) to a respective osseous fragment, the at least one sensor (4) constrained integrally to the main structure (2) and capable of detecting at least one mechanical parameter, the main structure (2) is at least partly made by a three-dimensional moulding process and the at least one sensor (4) is incorporated into the main structure (2) during the three-dimensional moulding process.

The present invention relates to a device for osteosynthesis of the type specified in the preamble to the first claim.

As is known, the treatment of osseous fractures can be conservative or surgical. In the first case, the osseous stumps are kept faced with the application of immobilisation devices such as plaster. The surgery is the treatment of choice for certain types of fractures that do not allow satisfactory clinical results to be obtained with immobilisation alone. The surgical act consists of repositioning the fractured osseous fragments either by exposing the fracture area or by external manoeuvres and immobilising them using special synthesis means. Furthermore, it is possible to distinguish between external fixation, obtained by means of synthesis media that reach the osseous through small skin incisions, and internal fixation, obtained by means of synthesis media attached to the osseous or positioned within it and therefore entirely covered by soft tissue. Specifically, internal means of synthesis can be intramedullary nails, plates, screws or others. Such devices are usually made of metal, preferably titanium alloy or stainless steel.

The fracture healing is a process that can take several weeks. The time for complete healing is highly variable depending on the patient and the presence of comorbidities. Following surgery, the patient requires continuous checks to monitor the healing progress of the fracture. These controls generally involve invasive procedures such as, for example, computed tomography, magnetic resonance imaging, radiography, or others. Although the aforementioned procedures are extremely useful for medical personnel to determine the progress of healing, they have certain disadvantages. First of all, some procedures result risky because the patient is exposed to X-rays.

Moreover, these techniques are generally expensive and/or complex, require advance planning and only give an image of the healing site at the time they are performed. After implantation, and during the healing process, the osteosynthesis devices are subject to various forces. The knowledge of the direction and magnitude of these forces can help the physician monitor the healing process without the need to subject the patient to X-ray examinations. Prosthetic devices that include at least one sensor are currently known. Such a sensor may be, for example, a pressure sensor as described in patent U.S. Pat. No. 8,661,893. In the cited patent, the pressure sensor is enclosed within two layers defining the prosthetic device. The prosthetic device must therefore have a cavity capable of housing the aforementioned sensor and possibly include an electrical circuit. The known technique described includes some important drawbacks.

In particular, fracture healing monitoring generally involves the use of invasive and/or risky techniques for the patient.

Furthermore, the monitoring procedures generally used only provide an image of the healing site at the time they are performed.

In this situation, the technical task underlying the present invention is to devise an osteosynthesis device capable of substantially obviating at least part of the aforementioned drawbacks.

In the context of said technical task, it is an important aim of the invention to obtain an osteosynthesis device that allows monitoring the healing of fractures without the use of invasive and/or dangerous procedures.

Another important purpose of the invention is to achieve an osteosynthesis device that is easy to manufacture.

The specified technical task and purposes are achieved by osteosynthesis device as claimed in the appended claim 1.

Preferred technical solutions are disclosed in the dependent claims.

The features and advantages of the invention are hereinafter clarified by the detailed description of preferred embodiments of the invention, with reference to the appended drawings, in which:

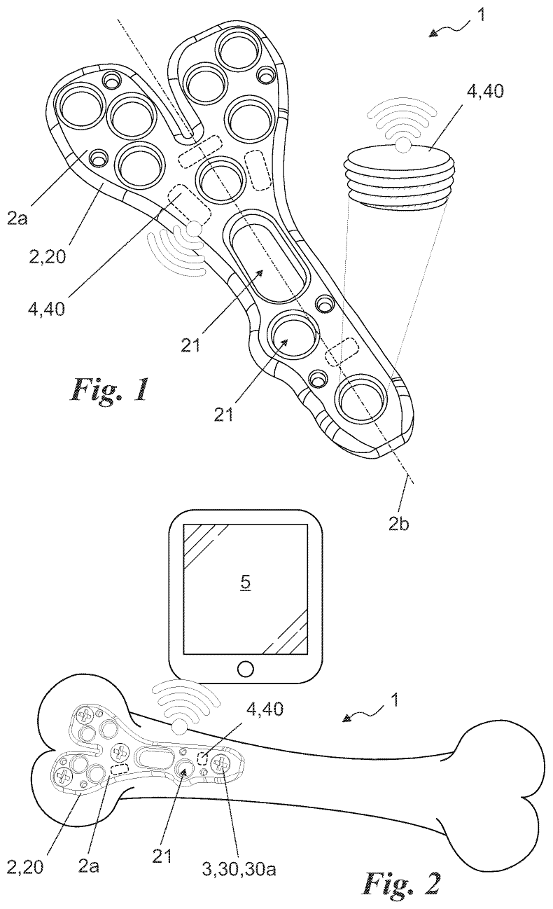

the FIG. 1 shows a perspective view of the osteosynthesis device according to the invention; and

the FIG. 2 illustrates the osteosynthesis device according to the invention applied to a fractured osseous.

In the present document, the measurements, values, shapes and geometric references (such as perpendicularity and parallelism), when associated with words like “about” or other similar terms such as “approximately” or “substantially”, are to be considered as except for measurement errors or inaccuracies due to production and/or manufacturing errors, and, above all, except for a slight divergence from the value, measurements, shape, or geometric reference with which it is associated. For instance, these terms, if associated with a value, preferably indicate a divergence of not more than 10% of the value.

Moreover, when used, terms such as “first”, “second”, “higher”, “lower”, “main” and “secondary” do not necessarily identify an order, a priority of relationship or a relative position, but can simply be used to clearly distinguish between their different components.

Unless otherwise specified, as results in the following discussions, terms such as “treatment”, “computing”, “determination”, “calculation”, or similar, refer to the action and/or processes of a computer or similar electronic calculation device that manipulates and/or transforms data represented as physical, such as electronic quantities of registers of a computer system and/or memories in, other data similarly represented as physical quantities within computer systems, registers or other storage, transmission or information displaying devices.

The measurements and data reported in this text are to be considered, unless otherwise indicated, as performed in the International Standard Atmosphere ICAO (ISO 2533:1975).

With reference to the Figures, the device for osteosynthesis according to the invention is collectively referred to as device 1.

The device 1 is configured to support the healing of an osseous fracture. In particular, it is suitable for use in cases where traditional immobilisation devices, for example plaster, do not provide satisfactory clinical results. In such cases, it is appropriate to resort to a surgical act that repositions the osseous fragments and immobilises them using devices for osteosynthesis such as device 1 described below.

The device 1 comprises, in brief, a main structure 2 and at least one sensor 4.

The main structure 2 defines an expansion surface 2a. In particular, the main structure 2 comprises a plate 20. The plate 20 extends predominantly along the expansion surface 2a.

The plate 20 may be flat. Alternatively, it may be slightly curved, preferably at one end. Such curvature is intended to improve the adaptability of the plate 20 to the surface of the osseous fragments. In detail, the plate 20 is preferably developed along a prevailing direction 2b. This plate 20, moreover, may be symmetrical with respect to the prevailing direction 2b. The plate 20 preferably has a thickness between 1.5 mm and 4 m. The plate 20 may have any shape and size depending on the type and conformation of the osseous fracture to be treated. The plate 20 is preferably substantially rectangular. More preferably it comprises, at one end, a portion arranged transverse to the prevailing direction 2b.

The plate 20 is advantageously made by a three-dimensional moulding process. In particular, it may be made of biocompatible plastic material. Alternatively, it may be made of metallic material, for example titanium alloy or stainless steel.

The plate 20 further comprises a plurality of through-holes 21. The through-holes 21 preferably pass evenly through the entire expansion surface 2a. Furthermore, the through-holes 20 may all have the same cross-section or they may have different cross-sections.

The through-holes 21 are suitable for accommodating, at least partially, fastening elements 3. The fastening elements 3 are capable of constraining integrally the plate 20 to a respective osseous fragment. Thus, the main structure 2, by means of the fastening elements 3, allows two or more osseous fragments to be locked in position and, if necessary, to be brought closer to each other so as to support the healing of said fracture.

The fastening elements 3 preferably comprise a plurality of screws 30. They may be fully or partially threaded depending on the type of fracture to be treated. The screws 30 are, in use, transverse to the expansion surface 2a. Furthermore, each screw 30 includes a head 30a which results, in use, at least partially aligned with the expansion surface 2a.

As anticipated above, the device 1 includes at least one sensor 4. Preferably, the device 1 includes a plurality of sensors 4. Preferably, the sensors 4 are integrally bonded to the main structure 2. Preferably, the sensors 4 are incorporated into the main structure 2 during the three-dimensional moulding process. In addition, one or more sensors 4 may be added later in the three-dimensional moulding process. For example, such sensors may be inserted into the through holes 21.

The number and the position of the sensors 4 in the main structure 2 depend on the type and conformation of the osseous structure to be treated.

The sensors 4 are suitable for detecting at least one mechanical parameter. Preferably, they are configured to detect the intensity and/or direction of tensile and compressive forces acting on the main structure 2. For example, such sensors 4 may comprise a piezoelectric element 40 which, upon mechanical deformation, generates a potential difference. Such a piezoelectric element may also be connected to an electrical circuit. The compressive and/or tensile forces acting on the main structure 2 thus generate a current variation in the electrical circuit. This current variation represents an electrical signal which can be detected by an external apparatus 5.

The external device 5 may, for example, be configured to detect a radio frequency electrical signal.

The operation of the device 1 described above in structural terms is as follows.

An operator places an external device 5 in the vicinity of an osseous fracture treated with a device 1. The external device 5 detects the signal generated by the sensors 4 included in the device 1. This signal provides information regarding the intensity and/or direction of the mechanical forces acting on the main structure 2. In this way, the operator can frequently monitor the healing process of a patient's osseous fracture. The invention comprises a new process. It consists in making a device for osteosynthesis 1 by means of a three-dimensional moulding process. During said three-dimensional moulding process, one or more sensors 4 are incorporated into the main structure 2 of the device 1.

The device 1 according to the invention achieves important advantages.

Indeed, the device 1 makes it possible to frequently monitor the healing process of osseous fractures without resorting to invasive or dangerous techniques for the patient. Furthermore, the device 1 is easy to manufacture as it is produced by three-dimensional moulding and the sensors 4 are incorporated into the device 1 during such three-dimensional moulding.

The invention is susceptible to variations within the scope of the inventive concept as defined by the claims.

Within this scope, all details are substitutable by equivalent elements and the materials, shapes and dimensions can be any.

1. Device (1) for osteosynthesis configured to support the healing of an osseous fracture comprising:
a main structure (2) defining an expansion surface (2a) and comprising:
a plate (20) extending mainly along said expansion surface (2a),
fastening elements (3) capable of integrally constraining said plate (20) to a respective osseous fragment,
at least one sensor (4) integrally constrained to said main structure (2) and capable of detecting at least one mechanical parameter,
and characterized by:
said main structure (2) is at least in part made by means of a three-dimensional moulding process and said at least one sensor (4) is incorporated in said main structure (2) during said three-dimensional moulding process.

2. Device (1) according to claim 1, wherein said plate (20) comprises a plurality of through holes (21) capable of housing at least partially said fastening elements (3).

3. Device (1) according to claim 1, wherein said fastening elements (3) comprise a plurality of screws (30) each including a head (30a).

4. Device (1) according to claim 3, wherein said screws (30) are, in use, arranged transversely to said expansion surface (2a) and said head (30a) is at least partially aligned with said expansion surface (2a).

5. Device (1) according to claim 1, wherein said at least one sensor (4) is configured to detect tensile and compression forces acting on said main structure (2).

6. Device (1) according to claim 1, wherein said at least one sensor (4) comprises a piezoelectric element (40).

7. Device (1) according to claim 6, wherein said piezoelectric element (40) is capable of generating an electrical signal detectable from an external device (5).

8. Device (1) according to claim 1, comprising a plurality of said sensors (4).

9. Device (1) according to claim 1, wherein said plate (20) is made of biocompatible plastic material.

10. Process for manufacturing a device (1) according to claim 1, characterized by comprising the phases of:
forming said main structure (2) by means of a three-dimensional moulding process, and
incorporating said at least one sensor (4) in said main structure (2) during said forming phase.
